(19) United States
(12) Patent Application Publication
(10) Pub. No.: US 2021/0183059 A1
(43) Pub. Date: Jun. 17, 2021

(54) IMAGE ACQUISITION METHOD USING A COLOR TRANSFORMATION AND ASSOCIATED MEDICAL IMAGE ACQUISITION SYSTEM

(71) Applicant: SCHÖLLY FIBEROPTIC GMBH, DENZLINGEN (DE)

(72) Inventors: ANDREAS HILLE, VILLINGEN-SCHWENNINGEN (DE); SÉBASTIEN WEITBRUCH, NIEDERESCHACH (DE); JONATHAN SCHULZ, LÖFFINGEN (DE)

(73) Assignee: SCHÖLLY FIBEROPTIC GMBH, DENZLINGEN (DE)

(21) Appl. No.: 17/123,634

(22) Filed: Dec. 16, 2020

(30) Foreign Application Priority Data
Dec. 17, 2019 (DE) 102019134799.8

(51) Int. Cl. G06T 7/00; G06T 7/90; G06T 7/11; G06T 11/00; A61B 1/00

(52) U.S. Cl. CPC G06T 7/0012; G06T 7/90; G06T 7/11; G06T 2207/10068; A61B 1/00009; G06T 2207/10024; G06T 11/001

(57) ABSTRACT

Medical image acquisition method for improving the identification of objects using characteristic colors in a color image which has been captured with an image sensor of a medical image acquisition system, wherein firstly a color value of an image area, selected by a user, of the color image is determined at least partially in a computer-implemented manner, and that subsequently, based on the determined color value, a color transformation is applied to the color image, which increases the color distance between image areas of the color image which are identical or similar in color to the determined color value and remaining image areas, not similar in color, of the color image.

### CROSS-REFERENCE TO RELATED APPLICATIONS

This application claims priority to German Patent Application No. 102019134799.8, filed on Dec. 17, 2019, which is herein incorporated by reference in its entirety.

### FIELD OF THE INVENTION

The invention relates to an image acquisition method in which a sequence of color images is captured with an image sensor of a medical image acquisition system (for example of an endoscopy system, of a digital microscope or of an exoscope), wherein at least one of the color images of the sequence is subjected to a color transformation to generate a desired representation of the color image on a monitor. Here the sequence may be recorded as a series of still images and/or, in particular, it may be recorded as a video image data stream.

In addition, the invention relates to a method for recording a sequence of color images.

Lastly, the invention relates to an associated medical image acquisition system, which comprises an image sensor and preferably also a camera control unit.

### BACKGROUND OF THE INVENTION

Such methods are known and are used, for example, during surgical procedures which are monitored by a video endoscope as they are performed. In particular, these procedures include operations in which a specific malignant tissue inside the patient's body is stained with the aid of dyes administered to the patient to be able to distinguish these tissue sections from healthy tissue and remove them by surgical means accordingly. In this context one refers to a ‘chromoendoscopy’, as the malignant tissue is identified with the aid of an endoscope and using a chromophore provided by the dye.

To this end, dyes such as methylene blue or indigo carmine are sprayed on a tissue to be examined to thereby, in particular, be able to display changes to mucous membranes with increased color distance, i.e. through highlighting in color, and in particular to be able to identify malignant changes to the tissue.

Until now, this identification has been performed by the surgeon himself, who, using individual or consecutive images from the recorded image sequence, recognizes the malignant tissue on the basis of a characteristic hue (color) of the chromophore. Here the surgeon faces the task, in what each time is a complex ‘scenery’ of an image captured by an endoscope, of identifying—as far as possible—all the tissue sections in the relevant image that display the characteristic hue of the dye used, for example dark blue in the case of methyl blue, so as to thereby remove all of the malignant tissue as completely as possible. This selection of the malignant tissue in the particular color image on the basis of the hue is challenging for the surgeon, particularly because the selection and/or identification are heavily dependent on the quality of the image representation, the specific scene and on the illumination settings.

### SUMMARY OF THE INVENTION

The task of the invention is to propose a method which assists the surgeon with this selection. In particular, the error rate ought to be reduced compared with current methods.

To solve this task, in accordance with the invention the features of Claim 1 are provided in an image acquisition method. Thus in particular, in accordance with the invention and to solve the task in the case of an image acquisition method of the type described at the beginning, it is proposed that in one of the color images of the sequence an image area is selected, a color value is determined for the selected image area, and that—on the basis of the determined color value—the color transformation is applied in such a way that a distance in color (=color distance) between the determined color value and remaining color values of non-selected image areas of the at least one color image is increased.

Here, the color image in which the image area, which is used to determine the color value, is selected may be in particular a first color image of the image sequence.

Here the term color value may be used for example to refer to a hue value, a color saturation value (e.g. a “saturation value”) or a color brightness value. However, color values can also be obtained which are calculated from a combination of such color parameters, for instance a color value which takes into consideration both color saturation values and hue values.

The color images from the image sequence may, in addition, reproduce either identical or different scenarios. The latter will be the case for example if the image sequence is recorded continuously in the form of a video image data stream.

The image sequence may also, for example, consist of a first still image and an associated video image data stream. Here the still image may have been obtained from the video image data stream or have been recorded separately from the video image data stream. In addition, the image area may be selected for determining the color value in the still image, and the color transformation may be applied to the color images from the video image data stream.

The at least one color image, to which said color transformation is applied, may therefore be a single color image from the color sequence or a plurality of color images, in particular, therefore, a specific sequence of color images.

In other words, the invention proposes highlighting in color a selected image area—using its determined color value—in comparison to other, non-highlighted areas, by increasing a color distance between the determined color value of the selected image area and color values within non-selected image areas which deviate from the determined color value. As a result, the method thus increases the color distance perceptible to a user between the selected image area and the non-selected image areas. At the same time, the non-selected areas have color values which deviate from the determined color value of the selected image area and are dissimilar to this in color.

By increasing the color distance, it is therefore possible for a computer-assisted, visual differentiation of selected and non-selected image areas to take place. As will be explained more precisely, the increase in said color distance may in particular occur through manipulation of hue values, color saturation values, or perhaps color brightness values.

The advantage of this method is firstly that a color difference or color distance between the selected image area and remaining image areas of the color image that is already detectable by the human eye can be hugely increased. By this means, firstly the perception of the selected image area is improved.

As will be shown more precisely later on, it is additionally possible to extend the color distance increase also to further image areas which are close in color to the selected image area. By this means, image regions that are related, color-wise, can be better distinguished from regions that are further removed in terms of color. As was explained at the start, the practical benefit of this technical effect in medical applications lies in an improved differentiation of tissue that is only slightly different in color and thereby, ultimately, in safer diagnostics.

The invention is applicable to all commonly used image acquisition methods; alongside image acquisition methods based on dyes for staining tissue, these also include fluorescence-based image acquisition methods in which, with the aid of a fluorophore and using stimulation light specially tailored to the particular fluorophore, a characteristic hue is generated.

A color transformation according to the invention may be understood, in particular, as a transformation of image data between standardized color spaces, for example from the RGB color space to the HSV color space. With such a transformation, color values such as hue values, color brightness values and color saturation values are typically retained unchanged.

In addition, however, with a color transformations according to the invention, such values may also be changed to improve the display of the color image on the monitor for a particular application, for example for the situation of a medical operation. Hence the color transformation may, in particular, comprise a processing and modification of color values, in particular in the form of color coordinates. As a result, the color transformation therefore leads to an increase in said color distance.

The color transformation may for example be performed by a camera control unit, more precisely in a FPGA of a camera control unit, of a medical image acquisition system (so for example of an endoscopy system, a digital microscope or an exoscope) with the aid of which the method according to the invention is applied. To this end, the color transformation to be performed by the FPGA may be stored in corresponding storage cells, for example registers, meaning that the method can be carried out in an automated manner.

Segmentation of the color image into color segments using recognized objects, i.e. image structures, constitutes a further possibility for increasing the color distance of individual structures within the color image. However, compared with this approach, the invention has the particular advantage that, specifically, renewed identification of the object highlighted in color is not necessary. Rather, the method according to the invention directly meshes with color processing i.e. image processing of the color image captured with the image sensor, and does so independently of image content actually displayed.

These advantages are particularly evident when the shape of the object, which is monitored with the image sensor, changes, for example when parts of a tissue are removed or because the shape of the tissue changes. Such a change in the captured scene may however also arise from, for example, simply zooming in and zooming out.

In all these cases, while the image structures change, the increase in the color distance provided by the method according to the invention is retained, because it is based on the identification not of image structures but of color values. This shows the robustness of the method according to the invention.

In addition, the method according to the invention can, after a single determination of the color value of the selected image area of a single image, be applied successively to a sequence of color images, for instance from a video image data stream captured with an image sensor, without having to re-identify certain structures each time. As a result, the requirements regarding any necessary computing power for carrying out the method are minor compared with methods based on structure identification which needs to be constantly repeated.

In accordance with the invention, the task can also be solved by means of other advantageous designs as per the dependent claims.

For example, the aforementioned color distance between the determined color value and remaining color values of non-selected image areas of the at least one color image may be for example a color saturation value distance, in particular a distance of ‘S’ values. As will be explained more precisely, the color distance can consequently be increased by adapting color saturation values of the selected image area and/or of the remaining non-selected image areas, taking into consideration the determined color value, in particular a determined color saturation value. For example, the color distance may be increased by increasing color saturation values of the selected image area and/or by reducing color saturation values of the non-selected image areas; here the reverse procedure (lowering of color saturation values of the selected image area and/or raising of color saturation values of the non-selected image areas) is also possible, but it is not recommended, due to the poorer detectability of the selected image areas.

In an analogous manner, the color distance may also be a hue value distance. Thus the color distance may be increased by adapting hue values of the selected image area and/or of the remaining non-selected image areas, taking into consideration the determined value, in particular a determined hue value. For example, the color distance can be increased by increasing hue values of the selected image area and/or by reducing hue values of the non-selected image areas; again the reverse procedure is also possible.

Ultimately, the color distance may also be a color brightness value distance, so for example a distance of ‘V’ values. Hence the color distance can be increased by adapting color brightness values of the selected image area and/or of the remaining non-selected image areas, taking into consideration the determined color value, in particular a determined color brightness value. For example, the color distance can be increased by increasing color brightness values of the selected image area and/or by reducing color brightness values of the non-selected image areas; here too, the reverse procedure is also possible.

Ultimately, these three approaches to increasing the color distance can also be used in combination (cf. in particular the exemplary embodiment according to FIG. 9). Consequently the color transformation may result in an increase in a color saturation value distance and/or a hue value distance and/or a color brightness value distance. This may in each case relate to a comparison between the selected image area and the non-selected image areas.

The aforementioned image area may preferably be selected using a characteristic hue, in particular by a user or in a computer-assisted or computer-implemented manner. At the same time, the characteristic hue may for example be generated using a dye with which it is possible to stain tissue. Or, the characteristic hue is a natural hue of a tissue, in particular a malignant tissue. That means that the method according to the invention can particularly be used in chromoendoscopy.

For a user-friendly design of the method, it is particularly beneficial if a user is shown, on a monitor, the image area that he must select and/or he has already selected. This is because by this means the user is able to check his selection decision and to correct it if necessary.

Using the determined color value, additional image areas of the at least one color image to which the color transformation is to be applied, in particular such image areas which likewise have the determined color value, can be selected automatically. These further image areas hence form part of the originally non-selected image areas of the color image. Through this automatic selection, a great burden is taken off the user, because it is now possible, without spending more time on it, to also select image areas that have the determined color value but are difficult to recognize.

It is exceptionally advantageous in this case if the color transformation for increasing the color distance is applied to the automatically selected, additional image areas. This is because, by this means, these additional image areas become easier for the user to recognize and therefore to check, because they appear uniform compared with the image area, selected by the user, to which the color transformation has already been applied. This approach can be applied for all three previously presented approaches to increasing the color distance. Hence the color transformation designed to increase the color distance, which is applied to the automatically selected, additional image areas, can accordingly adapt hue values and/or color saturation values and/or color brightness values of the automatically selected, additional image areas.

Here the automatically selected, additional image areas may in particular have additional image pixels with color values that deviate from the determined color value. This is because by means of, for example, averaging in each case, such image areas can also be automatically selected which potentially have no image pixels with precisely the color value determined but have image pixels which on average have high color similarity to the selected image area and thereby lie within a color similarity space in relation to the determined color value, as will be explained in more detail.

The determined color value may for example be determined using a statistical value, calculated from image pixels of the selected image area, i.e. in particular using a mean value. To this end, preferably RGB values of these image pixels may be processed.

In accordance with a specific embodiment, during the color transformation an output signal, in particular a raw data signal, from the image sensor, preferably in the form of a RGB signal, can be converted into a signal in a hue-based color space. Such a hue-based signal may for example be an HSV signal.

Here HSV color space may in particular refer to a color space in which colors are defined with the aid of three coordinates, specifically: hue value (English: hue=H), color saturation value (English: saturation=S) and color brightness value (English: value=V). Here the hue value may for example be indicated as a color angle on the color circle (for example 0° for red, 120° for green, 240° for blue), whilst the color saturation may attain values between 0% (=neutral gray), 50% (=poorly saturated color) and 100% (=fully saturated, pure spectral color) and the brightness value may lie in an interval between 0% and 100% (0% =no brightness, 100% =full brightness).

It is also known that, instead of the brightness value (V), it is also possible to use other parameters for representing color. Consequently, additional possible hue-based color spaces which can also be used in accordance with the invention are the HSL color space based on a relative brightness (L=lightness), the HSB color space based on an absolute brightness (B=brightness) and the HSI color space based on a light intensity (I=intensity).

In accordance with one preferred variant, the determined color value of the selected image area is a hue value, i.e. a hue value. This hue value may for example be averaged over image pixels of the selected image area.

It is also preferable if a color saturation value, in particular an average color saturation value, of the selected image area is determined using the signal in the hue-based color space, i.e. in particular using the HSV signal.

The aforementioned color transformation may hence occur giving consideration to the determined hue value and/or the determined color saturation value (cf. in particular the exemplary embodiment according to FIG. 9). Here, a particularly preferred embodiment of the method proposes that color saturation values of the at least one color image be adapted using the determined color saturation value by means of the color transformation.

For the final representation of the at least one color image on the monitor, the HSV signal obtained can, once the manipulation of the color saturation values and/or of the hue values and/or of the color brightness values of the color image has taken place, be subsequently transformed back into the RGB space.

At the same time, the manipulation of the color saturation values may preferably be performed using the determined saturation value of the selected image area. In an analogous manner, the manipulation of the color brightness values can take place using a determined color brightness value and/or the manipulation of the hue values can take place using a determined hue value.

To increase the color distance between the determined color value of the selected image area and the remaining color values of non-selected image areas of the at least one color image, there are at least three possibilities which can also be used in combination, as previously explained; specifically these are the manipulation of hue values, of color saturation values and of color brightness values.

Thus the color distance may for example be increased by raising a color saturation of the selected image area. Preferably, a color saturation of the automatically selected, additional image areas may also be raised at the same time. These elevations may preferably take place pixel by pixel in each case. An analogous approach may be applied with regard to hue values or color brightness values to increase the color distance.

The raising of the color saturation may for example take place through the increase—in each case—of an associated saturation value as a function of a saturation value determined for the selected image area. This determined saturation value may, in particular, be the average saturation value previously mentioned.

The color distance may also be increased by lowering a color saturation of the remaining non-selected image areas of the at least one color image. These non-selected image areas may in particular not have the determined color value and/or lie outside of a color similarity space of the determined color value, as will be explained more precisely.

Such a lowering may in particular be through reduction, in each case, of an associated saturation value as a function of a saturation value determined for the selected image area. This determined saturation value may likewise be the average saturation value mentioned above.

These described approaches, in other words the lowering of color values of the remaining non-selected image areas, are similarly applicable to hue values or color brightness values.

In summary, the color distance can be increased by raising color values of the selected image area, preferably and also of the automatically selected additional areas. Alternatively or additionally, it is also possible—for the same purpose—to lower color values of the remaining non-selected image areas, in particular such image areas that do not have the determined color value and/or lie outside of a color similarity space of the determined color value.

To enable a detailed display of image information, in spite of the color transformation, the color transformation may be designed such that it preserves respective brightness values and/or hue values of the selected image areas and/or of the remaining non-selected image areas of the at least one color image. This is particularly suitable when the color saturation values are adapted for increasing the color distance.

Alternatively, provision may also be made, in particularly if different color values—for instance color saturation values and hue values—are adapted, for the particular relative differences in color values in the selected image areas to be preserved. The same also applies to the non-selected image areas.

This is possible, for example, if the adaptation of the particular color value of all pixels of the selected areas occurs uniformly, i.e. by about the same amount. It is conceivable, for instance, to raise the color saturation values of all these pixels by a constant percentage (which corresponds to a shift of all color saturation values within the color similarity space) and/or to increase the hue values of all these pixels by a constant color angle (which corresponds to a rotation of the color similarity space).

For raising the color saturation of the selected area and in particular also of additionally automatically selected further image areas, it is proposed, in accordance with the invention, to raise the color saturation values of such image pixels, which have color values that are similar, in particular identical, to the determined color value.

In addition, for the purpose of lowering the saturation values of the non-selected areas, provision may for example be made to reduce the saturation values of image pixels of these areas percentagewise or set them at a specific low saturation value. This low saturation value ought ideally to be smaller than a saturation value determined for the selected area, in order to enable color highlighting of the selected area.

In accordance with a specific embodiment it is provided that, for the purposes of increasing the color distance, a color similarity space is determined around the color value determined for the selected image area. This works particularly well in a hue-based color space, for example in an HSV color space. Consequently the color similarity space may in particular be designed in a wedge shape, for example when in the HSV color space, around a particular Hue value, a wedge-shape/piece of cake-shaped part is formed from the cylindrical HSV color space which contains color saturation values of 0-100%, color brightness values between [0 . . . 1] and hue values in a particular color angle range, for instance [240°+/−1.5°].

If such a color similarity space is determined, it is possible, for image pixels of the at least one color image which lie within the color similarity space, to adapt, in particular raise, the relevant color values, i.e. in particular color saturation values and/or hue values. Additionally or alternatively it is also possible, for image pixels of the at least one color image which lie outside of the color similarity space, to adapt, in particular lower, the relevant color values, i.e. in particular color saturation values and/or hue values. Doing both, and in particular in combination, results in an increase in the color distance between selected and non-selected image areas.

The previously described process steps shall be briefly illustrated in simple form by means of formulae; here it is clear that additional executions of the method according to the invention naturally exist which result in different formulae:

In accordance with the following example, the determined color value of the selected image area may for example be a hue value, described below with the variable “Auswahl[Selection]”. In this case, pixels with color values “FarbeX[ColorX]”, which are dissimilar to the hue value “Auswahl[Selection]”, may be determined and their color saturation values reduced, e.g. through the instruction:

Finally, it is also possible to increase color distances within a color similarity space (Abs[Hue(Auswahl)−Hue(FarbeX)]<Threshold) around the determined color value of the selected image area, for instance through the instruction

IF Abs[Hue(Auswahl)−Hue(FarbeX)]<Threshold

THEN Hue(FarbeX)=Hue(Auswahl)+HueAbstand(FarbeX)×FactorHue

wherein FactorHue>>1 and wherein

HueAbstand(FarbeX)=Hue(Auswahl)−Hue(FarbeX)

stands for the distance in terms of hue values between a pixel with color value “FarbeX” within the color similarity space and the selected image area. By means of this last instruction, the color distances within the color similarity space are therefore increased.

In general it can therefore be stated that, in accordance with the invention, for the purpose of improving the highlighting of the color similarity space, it may in particular be provided that for image areas lying within the determined color similarity space, a respective color distance to the determined color value of the selected image area is increased by raising or lowering color values in each case. This raising/lowering may in turn relate to color saturation values (preferred), to hue values or to color brightness values. It is clear that lowering the particular color value makes sense if the particular color value is smaller than the determined color value of the selected image area; the converse applies accordingly, that an increase in the particular color value makes sense if the particular color value is bigger than the determined color value of the selected image area anyway.

The invention has also recognized that in particular when the color distance has been increased through the lowering of color values of the non-selected image areas, new color space is available which can be used for displaying the image areas to be highlighted. Consequently the invention proposes, among other things, that optionally all those image pixels, which lie within the determined color similarity space, may be selected as image pixels to be highlighted, and that the color similarity space may be subsequently elongated by extending color values of individual pixels out of those to be highlighted beyond the color similarity space. This is the case, for example, when a color value, for example a hue value/a hue value of an image pixel from a selected image area, is so heavily reduced or increased that the image pixel leaves the original color similarity space. By this means it is hence possible to use, in particular, colors/color saturation values which were not originally present in these image areas to display the image areas to be highlighted. The image areas to be highlighted may thereby become more colorful than they were originally and hence stand out even better from the non-selected image areas. The color similarity space described can be determined particularly easily in a hue-based color space, in particular in the HSV space, as will become clear by reference to the figures.

Here, a color saturation adaptation can be performed by calculating—for individual image pixels from the at least one color image—an absolute value (modulus) of a difference between a color value of the particular image pixel and the color value determined for the selected image area; by comparing the absolute value with a threshold value and, if the threshold value is exceeded, by increasing a color saturation value associated with the particular image pixel; and/or if the absolute value falls below the threshold value, by reducing a color saturation value associated with the particular image pixel. A similar approach can be used for hue value adaptation or for color brightness value adaptation; in these cases the differences refer to hue values or to color brightness values.

In addition, to further improve the representation, a color space of the at least one color image can be rotated such that the color value determined for the selected image area comes to rest on a nearest primary color, for example pure blue, pure red or pure green. The rotation may however also take place for example in such a manner that the color value determined for the selected image area comes to rest on what is the closest coming secondary color, for example pure cyan, pure magenta or pure yellow. Such colors are particularly eye-catching and therefore very easy for the user to distinguish.

Here the rotation of the color space may take place in such a way that the selected image area, and preferably also the automatically selected additional image areas, is/are displayed on the monitor in the primary color or secondary color. By this means, these areas relevant to the surgeon appear in a particular eye-catching manner.

In addition, the representation can be improved further by displaying the remaining non-selected image areas exclusively in color values other than the primary color. With this representation of the non-selected image areas, a color saturation can be used which, compared with a color saturation of the non-selected image areas, is reduced before the color transformation. In other words, for better representation it makes sense to lower the color saturation of the non-selected image areas so that these, when it comes to the overall impression of the at least one color image, recede into the background.

The color value to be determined for the selected image area of the color image may initially be automatically pre-determined by the medical image acquisition system. This may preferably occur by means of a statistical analysis of color values of the selected image area. The pre-determined color value may then be displayed to a user of the medical image acquisition system. Subsequently the user may confirm or discard the pre-determined color value, preferably via the monitor. Thereby, for example in medical applications, a relevant area of tissue can be identified very quickly by the user, selected and the associated color value determined semi-automatically.

Additionally or alternatively, provision may also be made for the user to be able to readjust a presented color value and/or a presented saturation value adaptation. Such measures allow the user to adapt the color value determination and the saturation value adaptation to his own requests in each case, as a result of which the user maintains precise control over the image acquisition technique.

Such readjustment may preferably take place with the aid of a color value scale and/or color saturation value scale displayed graphically on the monitor as an overlay. Here it is particularly preferred if the relevant scale has an additional fine adjustment scale for fine adjustment of the color value and/or saturation value.

In accordance with one embodiment, the user selects the image area manually on the monitor. In this case it is possible to subsequently, through image processing of the selected image area, determine the color value of the selected image area.

In another embodiment, which may be provided additionally or alternatively, the user is provided with a fixed target area for the computer-assisted selection of the image area. This can occur in particular by means of a graphic display, for example an on-screen-display (OSD), which is superimposed on the at least one color image displayed on the monitor. The fixed target area may preferably be provided by means of a graphic sight. By this means, the user can select the image area on the monitor with the aid of the target area.

The user may for example make the selection by shifting the desired image area through movement of the medical image acquisition system (so for example by moving an endoscope of an endoscopy system which uses the image acquisition method according to the invention) within the color image until the desired image area lies within the displayed target area. Subsequently the user may confirm the image area selection by waiting or by means of a confirmation action (tapping a GUI, a button or similar).

The color transformation may also be applied successively, preferably in real time, to multiple color images of the sequence. In particular, this may occur by increasing a color distance between the determined color value and non-selected image areas of the respective transformed color images.

Here it is particularly favorable if continuous adaptation of the relevant color distance takes place, in particular through continuous adaptation of color saturation values of the at least one color image.

In accordance with the invention, in order to determine the color value a determination of the selected area is only required in the case of one, in particular a first, of the color images of the sequence. This is because for example an image area selected at the beginning of the sequence and a color value determined once for this selected image area can be retained in the course of the sequence. The same applies to the adaptation of the color distance.

In yet other embodiments, although the determined color value can be retained in the course of the sequence, the adaptation of the color distance can be adapted through the color transformation in the course of the sequence, for example on the basis of a readjustment of the applied color transformation by means of a user entry.

It goes without saying that the image area selected at the beginning of the sequence may change during the recording of the sequence; for example, the selected image area can, for instance, change its position within the color image, for example if the medical image acquisition system (in particular the aforementioned endoscope) is moved; or the image area itself may change if, say, objects are to be introduced into an object area corresponding to the selected image area or if, for instance, tissue is moved in this object area.

By means of the method according to the invention, it is possible in all these situations that the medical image acquisition system automatically maintains the—on one occasion—increased color distance between the selected image area and the remaining non-selected image areas of the color image also in subsequent color images of the sequence and also if the scene captured with the image sensor changes. This is because, in particular, the automatic selection of additional image areas using the determined color value, as described above, can be performed again and again in subsequent color images of the sequence. By this means, the—in total—selected image areas can be continuously updated within the relevant color image of the sequence. In other words, the image area of a current color image that is highlighted by means of an increased color distance can be continuously updated during capturing of the image sequence.

In order to solve the named task, it is also proposed in the case of a medical image acquisition system of the type described at the start that the image acquisition system, in particular the camera control unit, has a controller which is set up to carry out, in combination with an external monitor, an image acquisition method as previously described and/or according to one of the claims directed towards an image acquisition method. Such a controller may for example be in the form of a FPGA with a controlling microprocessor.

To be able to make full use of the advantages of the invention, it is preferred, if the controller is set up to perform a color transformation with which a color distance between a selected image area of a color image captured with the endoscopy system and non-selected image areas of this color image is increased. This increase or this color transformation may be implemented, in particular, as previously described using the method according to the invention. To this end, an additional image processing unit may also be provided and/or, in particular, a color temperature of a light source may be taken into consideration which is used to illuminate a scene to be captured with the image sensor.

As was already explained, the selection of the image area may in particular occur using a characteristic hue and/or on the monitor. Here the color transformation may be designed as previously described in relation to the method according to the invention.

The invention shall now be described in more detail using exemplary embodiments, but is not limited to these exemplary embodiments. Further embodiments of the invention can be obtained from the subsequent description of a preferred exemplary embodiment in combination with the general description, the Claims and the drawings.

In the following description of different preferred embodiments of the invention, elements which are the same in terms of their function are given the same reference numbers, even if their design or shape differs.

### BRIEF DESCRIPTION OF THE DRAWINGS

The following are shown by the figures:

FIG. 1 a first color image of an image sequence that has been captured with an endoscope,

FIG. 2 a further color image of the sequence, wherein the observed image segment has shifted,

FIG. 3 the color image from FIG. 2, following the superimposition of a fixed target area,

FIG. 4 the color image from FIG. 3, after this has undergone a color transformation,

FIG. 5 an illustration of the HSV color space,

FIG. 6 a cross section through the HSV color space at a particular brightness value;

FIG. 7 left: the color image from FIG. 2 and right: a particular color value within the HSV color space from FIG. 6,

FIG. 8 left: the color image from FIG. 4 and right: an illustration of the color transformation performed in the color image from FIG. 4 within the HSV color space,

FIG. 9 an analogous representation to that in FIG. 8, wherein here the color transformation comprises an adaptation of color saturation values and of hue values, and

FIG. 10 the representation from FIG. 8, after elongating the color similarity space in which the selected image areas lay and

FIG. 11 a schematic diagram of an endoscopy system in accordance with the invention.

### DETAILED DESCRIPTION OF THE DRAWINGS

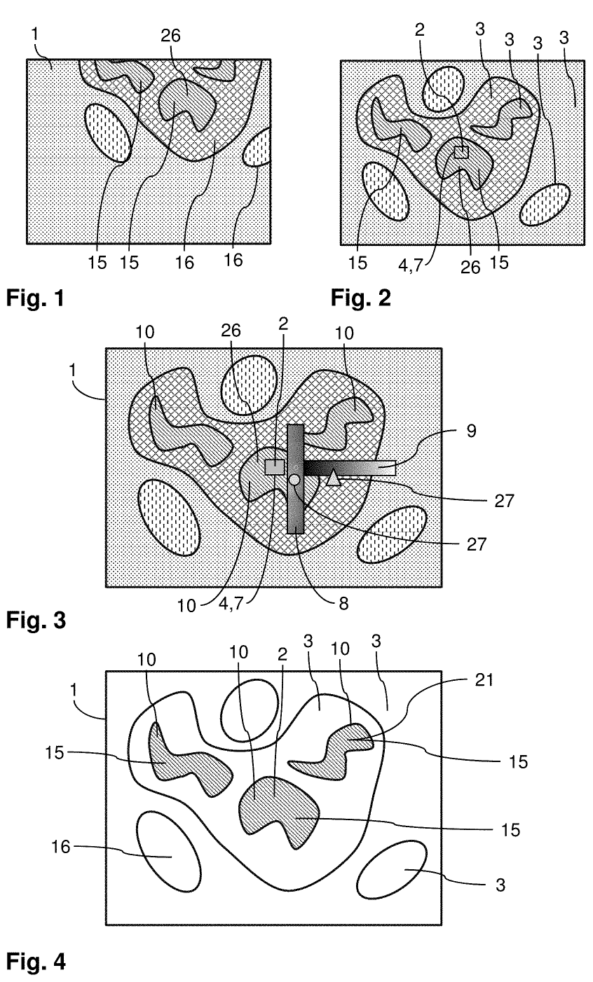

FIG. 1 shows a first digital color image 1 of a sequence of color images which was captured with an image sensor 23 of an endoscope of an endoscopy system, in other words a medical image acquisition system within the meaning of the invention. Color image 1 consists of a plurality of uniform image pixels and is observed by an surgeon as the user of the endoscopy system as a live video image on an external monitor 25. The video color image 1 reflects a typical endoscopic image acquisition situation during a surgical procedure. More specifically, in color image 1 of FIG. 1 both healthy tissue 16 and malignant tissue 15 can be identified. The malignant tissue 15 has been stained with the aid of methylene blue, which is illustrated by the hatching with solid lines.

FIG. 2 shows the same scene from FIG. 1, but recorded in a subsequent color image 1 of the sequence after the surgeon has moved the endoscope to shift a region of interest (ROI) 26 into the center of the image. The surgeon does this as long as the ROI 26 lies within a fixed target area 4 superimposed by means of a sight 7. After this, the surgeon operates a control key of the endoscopy system, by which means he selects the rectangular image area 2 which is defined by the target area 4 in the center of the image.

When making this selection, the surgeon is guided by the characteristic blue hue of the malignant tissue 15, i.e. he selects the image area 2 using the characteristic hue of the dye methylene blue with which the malignant tissue 15 has been selectively stained.

In addition the surgeon is shown on the monitor 25, with the aid of the target area 4, the image area 2 to be selected by him. After the selection, the target area 4 still remains superimposed and hence shows the surgeon the image area 2 selected by him at that particular time.

The selection of the image area 2 triggers a color value determination of the image area 2 manually selected by the surgeon. To this end, the color values, for example hue values, of all the image pixels which lie within the selected image area 2 are averaged in order to determine the color value 6 of the selected image area 2 as an average. To this end, an image processing unit of the endoscopy system processes RGB values of the image pixels within the selected image area 2.

The color value 6 of the selected image area 2 determined automatically by the endoscopy system through image processing and statistical analysis 6 is, in a subsequent step, firstly only displayed to the surgeon as a pre-determined color value 6. This takes place by means of the vertical value scale 8 illustrated in FIG. 3, which visually illustrates the pre-determined color value 6 to the surgeon with the aid of a display element 27.

In a next step, the surgeon can now accept the pre-determined color value 6 by operating the control key again.

He may also, however, firstly compare the color value 6 visually illustrated by means of the value scale 8 with the ROI 26. If, here, he identifies an unsatisfactory color deviation, he may with the aid of a fine adjustment scale 9—shown horizontally in FIG. 3—readjust the presented pre-determined color value 6. The display element 27 stylized as a triangle in FIG. 3 shows the surgeon what color value he has just set.

This fundamental approach for determining a color value can be applied to hue values, color saturation values and color brightness values.

If the surgeon has achieved, through readjustment, satisfactory matching between the now readjusted color value 6 and the ROI 26, he can then accept the color value 6 currently presented with the control key. With these steps, the surgeon has thereby, assisted by computer, determined the color value 6 of the image area 2 selected by him within the ROI 26 with high precision.

This high precision is significant for a subsequent step in which the endoscopy system, with the aid of the image processing unit, now automatically identifies additional image areas 10 within the color image 3 which—in terms of color—are very similar or even identical to the selected area 2.

The colors are identical if for example an image pixel shows the determined color value 6. The colors are similar, on the other hand, if an image pixel shows a color value which lies within a color similarity space 5 which was calculated around the determined color value 6 by the image processing unit using pre-set parameters or parameters readjusted by the surgeon. The color similarity space may for example take into consideration both hue values and color saturation values.

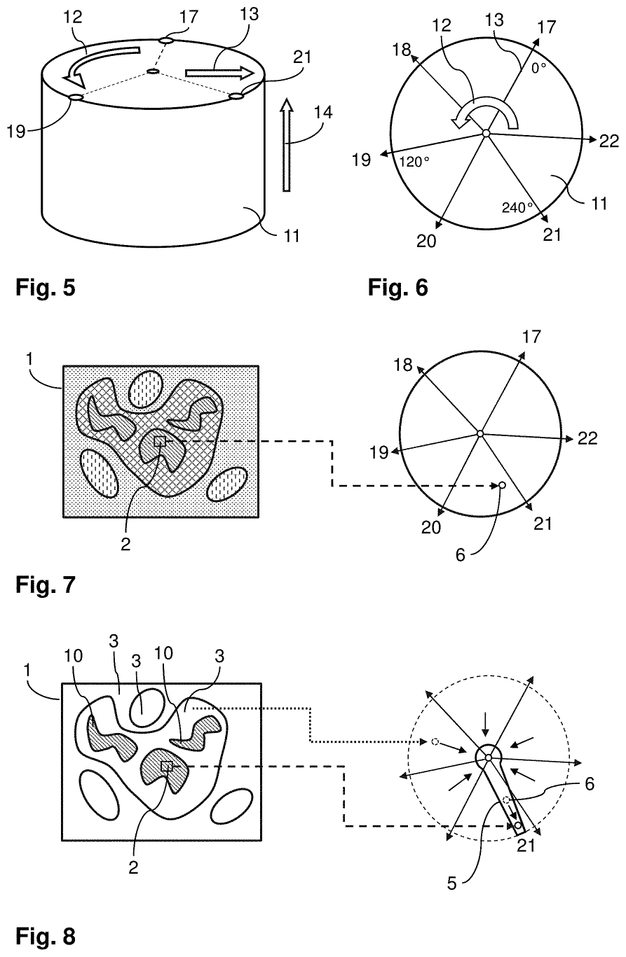

Using the color value 6 determined precisely by the surgeon and once readjustment of the color similarity space 5 has taken place, the endoscopy system now automatically selects the additional image areas marked with reference symbol 10 in FIG. 3. These additional image areas 10 have image pixels with color values that deviate from the determined color value 6. However, all of the pixels of the image areas now selected in total (and shown hatched in FIG. 4) lie in the color similarity space 5 with respect to the determined color value 6 of the image area 2 selected at the start (cf. FIG. 2), as will be explained more precisely using FIGS. 5 to 7.

In a final step, which is illustrated in FIG. 4, the endoscopy system now increases the color distance between the determined color value 6 of the selected image area 2 and the remaining color values of the non-selected image areas 3 of the color image 1 displayed in FIG. 4 by applying a color transformation to the entire color image 1. FIG. 4 can thus be viewed as being representative of a whole sequence of consecutive color images 1, which are captured successively with the image sensor 23 and to which said color transformation is successively applied.

This even extends so far that—if the surgeon moves the endoscope again—new additional image areas 10, which move into the color image 1 at the edge of the respective current color image 1, are again automatically selected by the endoscopy system, such that the color transformation is applied to these successively newly selected additional image areas 10. In simple terms, therefore, additional malignant tissue is successively highlighted in color by increasing the color distance, also when the surgeon actively changes the captured image scene by moving the endoscope. This repositioning of the color highlighting permits very simplified working, because a color highlighting of image areas selected using the determined color value, once set, can be successively extended to additional new image areas, if these new image areas are similar in color to the determined color value.

In the color transformation, an RGB signal 32 from the image sensor 23, which characterizes the particular color image 1 to be transformed, is initially converted into an HSV signal 40. After this RGB-to-HSV transformation 30, the image colors of all the image pixels of the particular color image 1 are no longer described by RGB coordinates but by HSV coordinates.

The color transformation, however, goes beyond pure coordinate transformation. This is because owing to the color transformation, a color saturation of the image area 2 selected by the surgeon at the beginning and of the additional image areas 10 automatically selected using the determined color value 6, all of which are shown hatched in FIG. 4, is subsequently raised. At the same time, a color saturation of the remaining non-selected image areas 3 of the color image 1, to which the transformation is applied, is lowered; as a result, these areas become pale in color and recede into the background.

Accordingly, the increased color distance that is recognizable in FIG. 4 between the selected image areas 2 and 10 and the remaining non-selected image areas 3 of the current color image 1 arises. These areas correspond, as a very good match, specifically to the malignant tissue 15 and the healthy tissue 16 respectively, as is visible in FIG. 4. As a result, on the monitor 25 the surgeon is therefore shown the malignant tissue 15 in the overall color image 1 as structures stained in dark blue with a high color distance from the remaining healthy tissue 16, which moves into the background as a result of the reduced color saturation.

The color transformation is set up in just such a way that the relevant brightness values and/or color values both of the selected image areas 2, 10 and also of the non-selected image areas 3 are retained. This has the effect that despite color highlighting, patterns and fine details remain recognizable to the surgeon in the whole color image 1.

For a better understanding of the concept of the invention, FIG. 5 illustrates the known HSV color space which has the three coordinates color angle 12 (or color value), color saturation 13, and brightness value 14, as illustrated by the arrows in FIG. 5. Each image pixel of the color image 1 may essentially reproduce a color which can be located within the HSV color space.

As FIG. 6 illustrates, primary colors such as red 17, yellow 18, green 19, cyan 20, blue 21 and magenta 22 correspond to the color angles 0°/60°/120°/180°/240°/300°.

The purer the relevant color, the higher its respective color saturation 13 and the further outside, in a radial direction, the associated color point lies within the color space 5 shown in FIG. 5. Here the representation of FIG. 6 constitutes a cross section through the HSV color space from FIG. 5, specifically for a constant brightness value 14.

Fundamentally, the (color) saturation describes how strongly a colored stimulus differs from an achromatic stimulus regardless of its brightness, in other words its distance from the achromatic axis (black-white axis), which specifically corresponds to the central axis of the cylindrical color space in FIG. 5.

Hence all hues can have a saturation of up to 100%, whilst white, grey and black show a saturation of 0% respectively.

As can be recognized in FIG. 7, the image processing unit has determined the color value 6 of the selected image area 2 within the HSV color space. To this end, as already previously described, firstly an average was calculated from RGB values of the image pixels of the selected image area 2 and subsequently this value transformed into the HSV space. Consequently the determined color value 6 is a hue value, averaged over image pixels of the selected image area 2. As can be recognized in the right-hand half of FIG. 7, the determined color value 6 lies in the vicinity of pure blue 21.

In the same manner it is possible, for the selected image area 2, to also determine an averaged saturation value which specifically corresponds to the radius of the pixel indicated with reference symbol 6 within the HSV space on the right-hand side of FIG. 7. Naturally, the same also applies to an average color brightness value which can also be determined for the selected image area 2 using averaging.

A first possibility for increasing the color distance on the basis of the color transformation performed by a controller of the endoscopy system is illustrated by the right-hand diagram in FIG. 8. There one can first recognize a wedge-shaped color similarity space 5 which is formed around the determined color value 6, i.e. calculated using pre-set parameters from the image processing unit.

For all pixels of color image 1 which lie within the calculated color similarity space 5, the color saturation was increased as part of the color transformation, which corresponds to a migration of these pixels outwards in a radial direction. Accordingly, clear, striking blue hues result for these image areas 2, 10. This can be clearly identified from the arrow directed outwards in a radial direction within the color similarity space 5, which indicates the increase in the color saturation value of the determined color value 6.

Since the color transformation is applied in an analogous manner to all pixels within the color similarity space, in particular also to pixels within the automatically selected additional image areas, there results an analogous increase in the color saturation values of all these pixels (not shown in FIG. 8). Here differences remain however, in the color saturation values and in the hue values, meaning that after the color transformation, too, structures remain recognizable within the image areas transformed by the color transformation.

For the remaining pixels, for example the pixel illustrated by means of the dotted arrow line, which lie outside of the color similarity space 5 and hence in non-selected image areas 3 of the color image 1, the color saturation has, by contrast, been lowered, which corresponds to a movement inwards in a radial direction in the HSV space and in FIG. 8 is illustrated by several arrows directed at the center of the HSV space.

After adapting the saturation values of the individual pixels/image pixels of the color image 1, the entire color image 1 was then transformed back again into the RGB space by means of an HSV-to-RGB transformation 29 and displayed on the monitor 25.

As has already been mentioned, all of these individual color transformation steps can be applied repeatedly to several successive color images 1 of the sequence.

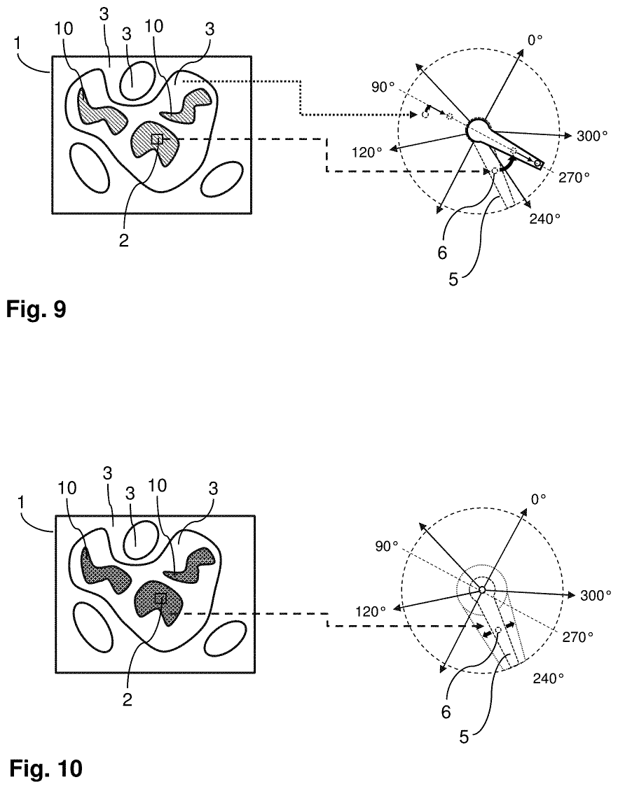

FIG. 9, which is designed in an analogous manner to FIG. 8, illustrates a further possible design of the color transformation which is used to increase the color distance: as can be recognized in the right-hand part of FIG. 8, firstly a color value 6 and a color similarity space 5 similar to the example from FIG. 8 were determined.

Subsequently, a color transformation was applied to all pixels within the color similarity spaces, including those of the automatically selected image areas. In order to increase the color distance firstly hue values, measured as color angles or hue values, were increased, which is indicated by the curved arrow on the right-hand side of FIG. 9. By this means, the hues of the respective pixels were thus increased by approx. 35°, so for instance for the determined color value from approx. 235° to 270°, which corresponds to a hue shift towards magenta (300°). This is clearly shown by the rotation of the color similarity space 5, as illustrated on the right-hand side of FIG. 9.

In a subsequent step, in addition to the hue shift, the color saturation values of the selected (including the automatically selected) image areas 2 were then increased and this was done analogously to the example of FIG. 8, which corresponds to a migration of the color values in a radial direction outwards in the right-hand picture in FIG. 9.

In addition, both of these steps were applied in reverse to pixels within the non-selected image areas. As is recognizable with the aid of the color value at approx. 95° color angle, to this end firstly the hue values of these pixels were reduced (which corresponds in the HSV space to a rotation in a clockwise direction, so for example from 95° to 90°, as illustrated on the right-hand side of FIG. 9) and subsequently their color saturation values were reduced (which in the HSV space corresponds to a migration inwards in a radial direction—cf. the radially inwardly directed arrow on the right-hand side of FIG. 9).

The first step of this two-stage color transformation may consequently be understood as a hue value elongation by means of which the color distances, measured in hue values, between selected and non-selected image areas is increased. By this means there may also be a hue shift of the selected area, meaning that this is displayed in a false color on the monitor after color transformation.

The color transformation was carried out both for the selected image areas 2 and for the non-selected image areas 3 in just such a way that the respective relative differences in the color values were preserved. As a result, the selected areas 2 and 10 shown hatched on the left-hand side of FIG. 9 appear in different colors following the transformation (shifted in the direction of magenta) and also with increased color saturation, but image structures are still recognizable in these areas because the relative differences in the hue values and the color saturation values between individual image pixels were preserved.

It goes without saying that an analogous color transformation also on the basis of color brightness values can be used alternatively or additionally to increase the color distance, as has already been explained above.

As a further optional step it is then possible—as shown in FIG. 10—for the color similarity space in which the selected image areas lie to be extended or elongated. To this end color values, in particular color saturation values but also hue values, of individual pixels of those which originally lay within the color similarity spaces, and which are to be highlighted in color (so the “image pixels to be highlighted”), may be extended beyond the color similarity space. As the HSV diagram in the right-hand part of FIG. 10 shows, for this purpose the color values, in particular hue values, of individual image pixels at the edge of the color similarity space are lowered/raised so much that they now lie outside of the original color similarity space, as shown by a comparison of the dotted with the dashed line. Through the elongation of the color space that takes place, the selected additional image areas 10 together with the originally selected image area 2 (both shown hatched in the left-hand diagram of FIG. 10) now cover a color space which, in terms of the hue values covers a larger color angle range than the original color similarity space 5. It should also be mentioned here that, in this case, color space which previously was occupied by non-selected image areas 3 is deliberately used to display the image areas 2, 10 to be highlighted. This is advantageous since the color values of the non-selected image areas 3, in particular their saturation values, were previously lowered meaning that these image areas 3, simply speaking, free up color space.

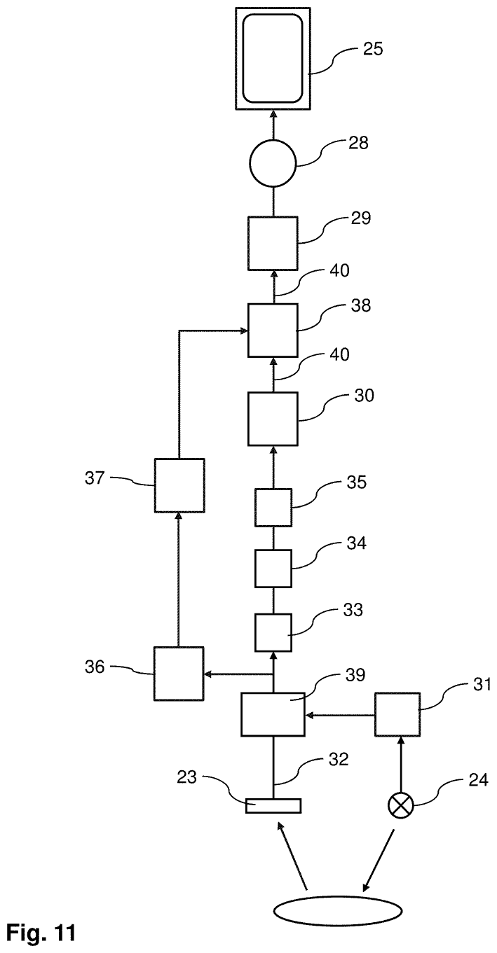

The diagram from FIG. 11 ultimately describes, schematically, the structure of an endoscopy system in accordance with the invention. This initially generates, with the aid of an image sensor 23, a sequence of color images 1 each of which are present as RGB image data 39. By means of a function “select image area” 36, in the RGB image data 39, firstly an image area 2 is selected by a user or by the endoscopy system itself for one of the color images and this is analyzed with an image processing system 37 in order to determine the color value 6.

After this, the entire color image 1 runs through a cascade of image processing steps such as edge filtering 33, noise filtering 34 and scaling 35, which help to improve the image quality before an RGB-to-HSV transformation 30 is applied to the color image 1, by which means an HSV signal 40 is generated. Using the color value 6—determined with the aid of image processing 37—of the selected image area 2, the color image 1 is then processed further in the HSV space, wherein the color distance is increased (=image processing in the HSV space 38—c.f. also the illustration on the right-hand side of FIG. 8 already outlined). In order to carry out this color transformation, use is made of a ‘color matrix’ which defines the transformation that is to be carried out.

This image processing in the HSV space 38 is followed by an HSV-to-RGB transformation 29, before the color image 1 is then transmitted as an RGB signal 32 by means of an on-screen display function 28 to the monitor 25 for display.

With this type of image processing or color transformation, the light source 24 that is used, or more precisely its color temperature, is taken into account. This is because the information from the light source 24 relevant to the image processing or the color matrix used in the process consists of the color temperature of the light emitted by the light source 24. The color temperature may for example be determined with the aid of a white fader. Also, in particular coefficients of the color matrix used for color transformation can be adapted on the basis of the determined color temperature of the light source 24.

In summary, in order to improve the recognition of objects using characteristic colors in a color image 1, which was captured with an image sensor 23 of a medical image acquisition system, it is proposed that firstly a color value 6 of an image area 2 of the color image 1 selected by a user is at least partially determined in a computer-implemented way and that subsequently, based on the color value 6 determined, a color transformation is applied to the color image 1 which increases the color distance between image areas 2, 10 of the color image 1, which are identical or similar in color to the determined color value 6, and other image areas 3 of the color image 1 that are not similar in color (cf. FIG. 8).

What is claimed is:

1. An image acquisition method comprising:
capturing a sequence of color images with an image sensor of a medical image acquisition system, and
subjecting at least one of the color images is to a color transformation to generate a desired representation of the at least one color image on a monitor, wherein the step of subjecting at least one of the color images to a color transformation comprises:
selecting an image area in one of the color images of the sequence;
determining a color value of the selected image area; and
applying a color transformation on the basis of the determined color value such that a color distance between the determined color value and remaining color values of non-selected image areas of the at least one color image is increased.

2. An image acquisition method in accordance with claim 1, wherein the color transformation results in an increase in a color saturation value distance and/or a hue value distance and/or a color brightness value distance,
in each case based on a comparison between the selected image area and the non-selected image areas; and
wherein the color distance is increased by adapting hue values and/or color saturation values and/or color brightness values of the selected image area and/or of the remaining non-selected image areas, taking into consideration the determined color value in each case.

3. An image acquisition method in accordance with claim 1, wherein the image area is selected using a characteristic hue, and
wherein the characteristic hue is generated by means of a dye with which tissue can be stained or that is a natural hue of a tissue, in particular a malignant tissue.

4. An image acquisition method in accordance with claim 1, wherein the image area to be selected by the user or already selected is displayed to the user on the monitor.

5. An image acquisition method in accordance with claim 1, further comprising:
automatically selecting additional image areas of the at least one color image, which have a determined color value;
applying a color transformation to the automatically selected additional image areas to increase color distance; and
wherein the automatically selected, additional image areas have additional image pixels with color values that differ from the determined color value.

6. An image acquisition method in accordance with claim 1, wherein the determined color value is determined using a statistical value calculated from image pixels of the selected image area, in particular an average; and
wherein, to this end, RGB values of these image pixels are processed.

7. An image acquisition method in accordance with claim 1, wherein in the color transformation an output signal, in particular a raw data signal, of the image sensor, preferably in the form of an RGB signal, is converted into a signal in a hue-based color space, in particular into an HSV signal; and
wherein the determined color value of the selected image area is a hue value, in particular averaged over image pixels of the selected image area; and
wherein a saturation value, in particular an average saturation value, of the selected image area is determined using the signal in the hue-based color space, in particular the HSV signal.

8. An image acquisition method in accordance with claim 1, wherein the color distance is increased by adapting, preferably raising, color values of the selected image area, preferably and of the automatically selected additional image areas, and/or by adapting, preferably lowering, color values of the remaining non-selected image areas of the at least one color image, which do not have the determined color value and/or lie outside of a color similarity space of the determined color value.

9. An image acquisition method in accordance with claim 1, wherein the color transformation preserves respective brightness values and/or color values of the selected image areas and/or of the non-selected image areas to enable a detailed representation of image information; or the color transformation preserves relevant relative differences in color values of the selected image areas and/or of the non-selected image areas.

10. An image acquisition method in accordance with claim 1, wherein in order to increase the color distance a wedge-shaped, color similarity space is determined around the color value determined for the selected image area; and
wherein, for image pixels of the color image which lie within the color similarity space, the respective color values, in particular color saturation values and/or hue values, are raised and/or for image pixels of the color image which lie outside of the color similarity space, respective color values, in particular color saturation values and/or hue values, are lowered.

11. An image acquisition method in accordance with claim 10, wherein for image areas lying within the determined color similarity space, a respective color distance to the determined color value of the selected image area is increased by raising or lowering color values in each case.

12. An image acquisition method in accordance with claim 10, wherein all image pixels which lie within the determined color similarity space are selected as image pixels to be highlighted; and
wherein the color similarity space is subsequently elongated by extending color values of individual pixels out of those to be highlighted beyond the color similarity space.

13. An image acquisition method in accordance with claim 10, wherein the color similarity space is determined in a hue-based color space, in particular in the HSV space, and/or a color saturation adaptation is performed by taking, for individual image pixels of the at least one color image, an absolute value of a difference between a color value of the respective image pixel and the color value determined for the selected image area; by comparing the absolute value with a threshold value; and by increasing a color saturation value associated with the relevant image pixel, if the threshold value is exceeded, and/or reducing a color saturation value associated with the respective image pixel, if the absolute value falls below the threshold value.

14. An image acquisition method in accordance with claim 1, wherein, to further improve the representation, a color space of the at least one color image is rotated in such a way that the color value determined for the selected image area comes to rest on a nearest primary color, for example pure blue, red or green, or on a nearest secondary color, for example pure cyan, magenta or yellow; and
wherein the selected image area and the automatically selected additional image areas, is/are displayed on the monitor in the primary color, while the remaining non-selected image areas are displayed exclusively in color values deviating from the primary color.

15. An image acquisition method in accordance with claim 1, wherein the color value to be determined for the selected image area is first automatically pre-determined by the endoscopy system by means of a statistical analysis of color values of the selected image area, and displayed to a user of the endoscopy system.

16. An image acquisition method in accordance with claim 15, wherein the user subsequently confirms or discards the pre-determined color value via the monitor, and/or re-adjusts a presented color value, and/or a presented saturation value adaptation, with the aid of a color value scale and/or saturation value scale shown graphically on the monitor as an overlay of an additional fine adjustment scale for fine adjustment of the color and/or saturation values.

17. An image acquisition method in accordance with one of the preceding claims, wherein a user selects the image area manually on the monitor such that the color value of the selected image area is determined, and/or the user is presented with a fixed target area for computer-assisted selection of the image area.

18. An image acquisition method in accordance with claim 1, wherein the color transformation is applied successively, preferably in real time, to multiple color images of the sequence, in a manner wherein a color distance between the determined color value and non-selected image areas of the respective transformed color images is increased.

19. A medical image acquisition system comprising:
an image sensor;
a camera control unit; and
wherein the image acquisition system includes a controller, such as an FPGA with a controlling microprocessor, which is set up to perform, in combination with an external monitor, an image acquisition method in accordance with claim 1; and
wherein the controller is set up for performing a color transformation with which a color distance between a selected image area of a color image captured with the image acquisition system and non-selected image areas of this color image is increased.
